(19) United States
(12) Patent Application Publication
(10) Pub. No.: US 2025/0052670 A1
(43) Pub. Date: Feb. 13, 2025

(54) PORTABLE INSTRUMENT FOR MEASURING GAS CONCENTRATION

(71) Applicant: HEALTHY PHOTON (NINGBO) TECHNOLOGY CO., LTD., NINGBO CITY (CN)

(72) Inventors: Yin WANG, Ningbo City (CN); Ting-jung LIN, Ningbo City (CN); Junhui ZENG, Ningbo City (CN); Jianwu ZHENG, Ningbo City (CN); Zhimei LIU, Ningbo City (CN)

(21) Appl. No.: 18/780,559

(22) Filed: Jul. 23, 2024

(30) Foreign Application Priority Data
Aug. 7, 2023 (CN) 202310983534.X

(51) Int. Cl. G01N 21/31; G01N 33/00

(52) U.S. Cl. CPC G01N 21/31; G01N 33/0027; G01N 2201/06113; G01N 2201/0636; G01N 2201/127

(57) ABSTRACT

Disclosed is a portable instrument for measuring gas concentration. A laser unit provides a laser beam required for laser spectral absorption. An optical unit includes a gas absorption cell (100). The gas absorption cell (100) includes a first reflective mirror (110), a second reflective mirror (120) and a cell tube (130), the first reflective mirror (110) and the second reflective mirror (120) are respectively connected to opposite ends of the cell tube (130). When the laser unit emits a laser beam, an optical path is formed between the first reflective mirror (110) and the second reflective mirror (120) within the cell tube (130). A heat recovery unit recycles heat generated by an electronic system unit (200) to the optical unit.

### CROSS-REFERENCE TO RELATED APPLICATIONS

This application claims priority to Chinese patent application No. 202310983534.X filed on Aug. 7, 2023, the disclosure of which is incorporated herein by reference in its entirety and for all purposes.

### TECHNICAL FIELD

The disclosure herein relates to the technical field of gas concentration measurement instrument, and specifically relates to a portable instrument for measuring gas concentration.

### BACKGROUND

Instruments for measuring gas concentrations are necessary in areas such as environmental protection, climate research, air quality assessment, and monitoring of some industrial processes.

In the context of modern environmental protection and climate change, the measurement and monitoring of greenhouse gases have become particularly critical. Common methods of measuring greenhouse gas concentrations include Gas Chromatography (GC), Non-Dispersive Infrared Absorption (NDIR), Laser Spectroscopy Absorption and so on. Each method has its merits and drawbacks. Gas Chromatography can measure a variety of gases but the instrument is expensive, and has high power consumption and slow measurement speed. Non-Dispersive Infrared Absorption is widely used in measuring carbon dioxide and methane concentrations, but its measurement accuracy is relatively low. Laser Spectroscopy Absorption has high measurement precision and fast speed.

The Herriott cell is an optical component based on Laser Spectroscopy Absorption, which has been widely used in gas analysis instruments. The Herriott cell increases the length of the optical path for interaction between gas and light through multiple reflections of light, thus improving the intensity and detection sensitivity of the absorption signal. However, existing Herriott cells cannot be applied in portable instruments for greenhouse gas measurement with low power consumption. On the other hand, the measurement accuracy of portable instruments with low power consumption often fails to meet the requirements of atmospheric observation.

### SUMMARY

One objective of the present disclosure is to provide a portable instrument for measuring gas concentration.

According to one aspect of the present disclosure, a portable instrument for measuring gas concentration comprising: a laser unit configured to provide a laser beam required for laser spectral absorption; an optical unit comprising a gas absorption cell, the gas absorption cell comprises a first reflective mirror, a second reflective mirror and a cell tube, the first reflective mirror and the second reflective mirror are respectively connected to opposite ends of the cell tube, when the laser unit emits a laser beam, an optical path is formed between the first reflective mirror and the second reflective mirror within the cell tube; an electronic system unit; a heat recovery unit configured to recycle heat generated by the electronic system unit to the optical unit.

Optionally, the heat recovery unit comprises a circulating water pipe, a section of the circulating water pipe is arranged adjacent to or in contact with the electronic system unit, and another section of the circulating water pipe is attached to or wound around the cell tube.

Optionally, the portable instrument for measuring gas concentration further comprising a closable carrier, wherein the optical unit, the laser unit, the electronic system unit and the heat recovery unit are all arranged in the closable carrier. When the closable carrier is closed, the optical unit, the laser unit, the electronic system unit and the heat recovery unit are all in an enclosed space.

Optionally, the closable carrier is a sealable carrier, and the enclosed space is an airtight space.

Optionally, the enclosed space is a non-airtight space.

Optionally, the closable carrier is provided with an air inlet and an air outlet, the air inlet or the air outlet is provided with a cooling fan, the closable carrier has a water cooling radiator arranged internally, the water cooling radiator is connected with the air inlet and the air outlet. Optionally, the cell tube is provided with a heating element.

Optionally, the laser unit is configured to provide at least two laser beams of different wavelengths; when the laser unit emits at least two laser beams, at least two separate optical paths are formed between the first reflective mirror and the second reflective mirror within the cell tube.

Optionally, the laser unit includes a first laser and a second laser, the first laser is positioned adjacent to the first reflective mirror or the second reflective mirror and is capable of emitting a laser beam along one optical path into the cell tube, the second laser is positioned adjacent to the first reflective mirror or the second reflective mirror and is capable of emitting a laser beam along another optical path into the cell tube.

Optionally, the first reflective mirror and the second reflective mirror are both provided with a first reflective portion and a second reflective portion distributed in concentric circles, the first reflective portion of the first reflective mirror and the first reflective portion of the second reflective mirror are composed of a plurality of first reflective points arranged in a ring, the second reflective portion of the first reflective mirror and the second reflective portion of the second reflective mirror are composed of a plurality of second reflective points arranged in a ring; the first reflective portion of the first reflective mirror and the first reflective portion of the second reflective mirror are arranged correspondingly, the laser beam emitted by the first laser is reflected back and forth between the first reflective portion of the first reflective mirror and the first reflective portion of the second reflective mirror to form the first optical path; the second reflective portion of the first reflective mirror and the second reflective portion of the second reflective mirror are arranged correspondingly, the laser beam emitted by the second laser is reflected back and forth between the second reflective portion of the first reflective mirror and the second reflective portion of the second reflective mirror to form the second optical path.

Optionally, the first reflective mirror or the second reflective mirror is provided with a first light entrance hole, and the first reflective mirror or the second reflective mirror is provided with a first light exit hole, the first reflective mirror or the second reflective mirror is provided with a second light entrance hole, and the first reflective mirror or the second reflective mirror is provided with a second light exit hole. The laser beam emitted by the first laser enters the cell tube from the first light entrance hole to form the first optical path and exits from the first light exit hole. The laser beam emitted by the second laser enters the cell tube from the second light entrance hole to form the second optical path and exits from the second light exit hole.

Optionally, the optical unit further comprising: a first detector located on an optical path of the laser beam exiting from the first light exit hole; and a second detector located on an optical path of the laser beam exiting from the second light exit hole.

Optionally, the portable instrument for measuring gas concentration further comprises a calibration unit. The calibration unit comprises an air pump and a pressure controller, the air pump is connected to the interior space of the cell tube through a pipeline and can inflate or deflate the cell tube, the pressure controller is arranged on the pipeline and configured to control an air pressure inside the cell tube.

Compared to existing technologies, some embodiments of the disclosure may produce the following advantageous effects.

First, through a thermal recovery unit, the heat generated by the subsystem units is collected and reused, which reduces the overall power consumption of the instrument, and ensures temperature stability of the instrument thereby improving the measurement accuracy of the instrument.

Second, a variety of gases can be concurrently measured with high precision through a single gas absorption cell, simplifying the instrument's structure and enhancing its portability.

Third, two optical pathway systems can be simultaneously formed within a single Herriott cell and two types of lasers are acceptable for simultaneous measurement of multiple gases. Thus, it eliminates the necessity for an abundance of equipment, resolves the issue of cumbersome equipment, and further enhances the portability of this instrument.

Fourth, the cell tube itself is equipped with a heating element. If the heat recovered into the cell tube is insufficient to heat the gas in the cell to a required temperature, the heating element can be used to control the gas temperature in the cell tube.

Fifth, when water passes by the electronic system unit, it provides an effect of water cooling and heat dissipation. The temperature of the water in the circulating water pipe increases because of heat absorption. As the water flows to the section of the circulating water pipe that is attached to or wound around the cell tube, the gas inside the cell tube can be heated by the recovered heat. This not only solves the problem of cooling the electronic system unit but also the issue of heating the gas inside the cell tube, thereby reducing the power consumption of both partial systems.

Sixth, the carrier can be a portable suit case, or a box, or a container. The presence of the carrier not only further enhances the portability of the entire instrument, but also provides an effect of being enclosed for all units, isolating them from the influence of the ambient temperature, dust, etc. Therefore, due to its portability, low power consumption, and being enclosed, the instrument of the disclosure can be used in outdoor or field environments.

Seventh, when calibration is required, the pressure inside the cell tube is set to zero through a pressure controller, and the air pump vacuums the cell tube, so that there is no gas molecule exit inside the cell tube in theory, and the gas concentration is proximate to or equal to zero. At this point, a calibrated zero point can be obtained by measurement taken by the optical unit.

### BRIEF DESCRIPTION OF FIGURES

FIG. 1 is a schematic diagram of the structure of the instrument of the disclosure.

FIG. 2 is a top view of the instrument of the disclosure when the carrier is open.

FIG. 3 is a schematic diagram of the structure of the carrier of the disclosure.

FIG. 4 is a schematic diagram illustrating the working principle of the optical unit of the disclosure.

FIG. 5 is a schematic diagram of the light spot points of the first reflective mirror of the disclosure.

FIG. 6 is a schematic diagram of the light spot points of the second reflective mirror of the disclosure.

FIG. 7 is a schematic diagram of the structure of the gas absorption cell of the disclosure.

### REFERENCE NUMBERS IN THE DRAWINGS

100: gas absorption cell; 110: first reflective mirror; 111: first light entrance hole; 112: first light exit hole; 120: second reflective mirror; 121: second light entrance hole; 122: second light exit hole; 130: cell tube; 140: first detector; 150: second detector; 160: heating element; 200: electronic system unit; 310: first laser; 320: second laser; 330: first optical path; 340: second optical path; 350: first reflective point; 360: second reflective point; 400: circulating water pipe; 500: carrier; 510: air inlet; 520: air outlet; 530: cooling fan; 540: water cooling radiator; 610: air pump; 620: pressure controller.

### DETAILED DESCRIPTION

The following are specific embodiments of the present disclosure, further describing the technical solutions of the present disclosure in conjunction with the accompanying drawings, but the present disclosure is not limited to these embodiments.

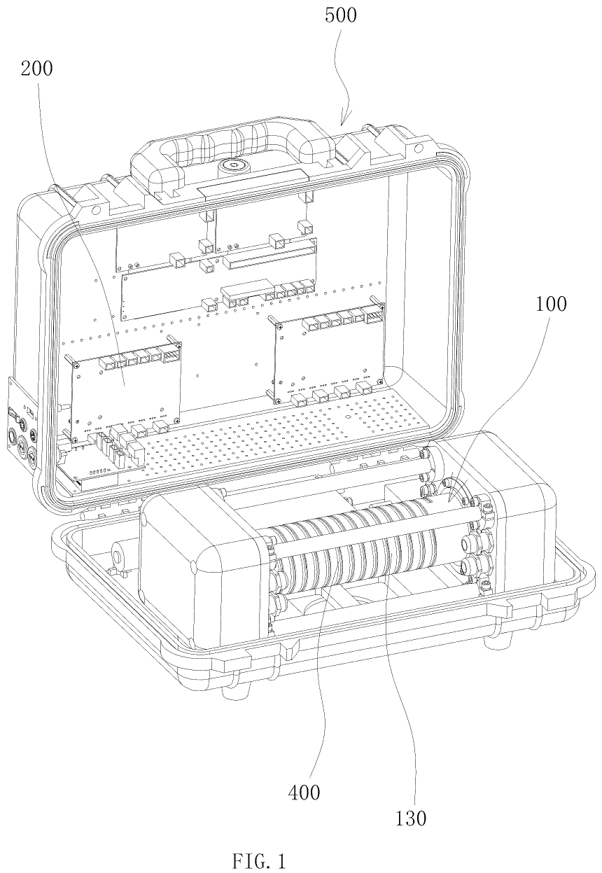

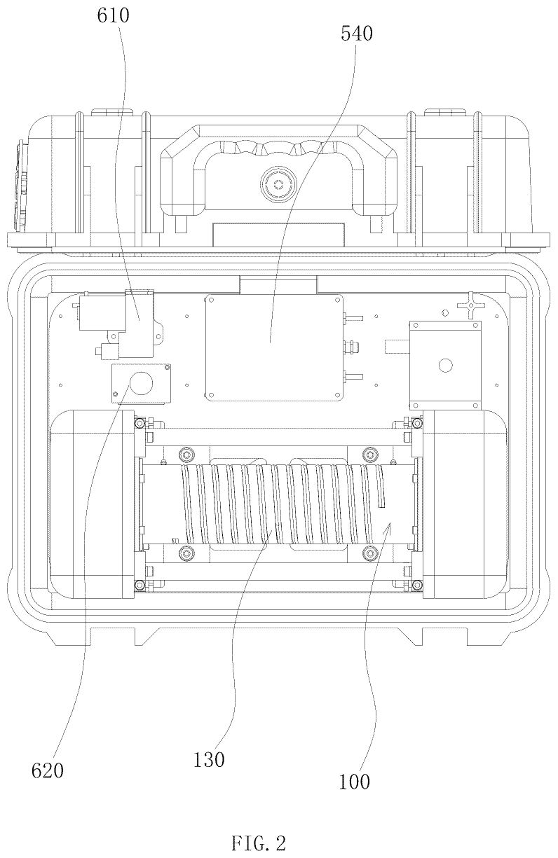

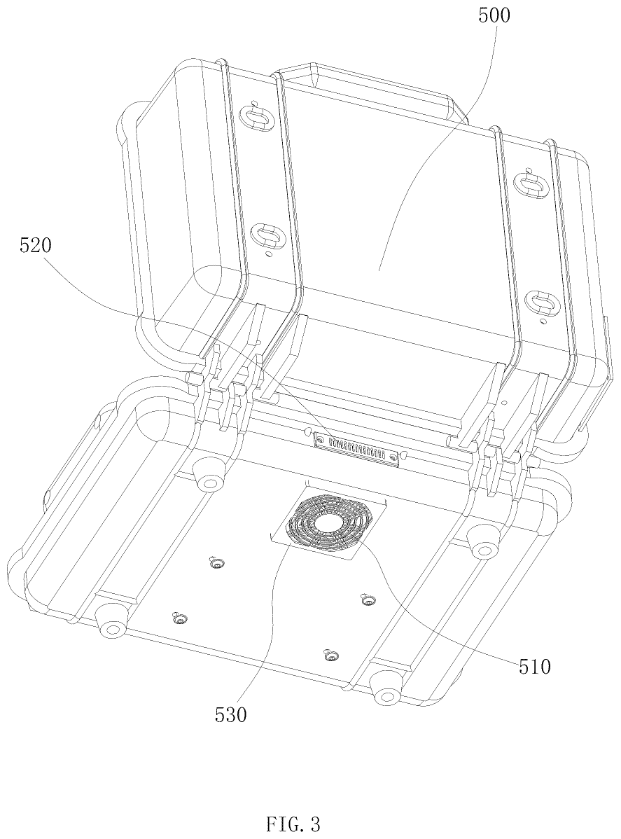

As shown in FIGS. 1-7, a portable instrument for measuring gas concentration with low power consumption includes a laser unit, an optical unit, an electronic system unit 200 and a heat recovery unit.

The laser unit can provide a laser beam. The optical unit includes a gas absorption cell 100, which may also be referred to as “gas absorption chamber”. The gas absorption cell 100 includes a first reflective mirror 110, a second reflective mirror 120 and a cell tube 130. The first reflective mirror 110 and the second reflective mirror 120 are respectively connected to opposite ends of the cell tube 130. When the laser unit emits a laser beam, an optical path is formed between the first reflective mirror 110 and the second reflective mirror 120 within the cell tube 130. A heat recovery unit is used to recycle heat generated by an electronic system unit 200 to the optical unit.

The laser unit is capable of providing a laser beam required for laser spectral absorption.

In some embodiments, the laser unit is capable of providing at least two laser beams of different wavelengths for measurement of multiple kinds of gases. In this case, at least two separate (or independent) optical paths are formed between the first reflective mirror 110 and the second reflective mirror 120 within the cell tube 130, when the laser unit emits at least two laser beams. Traditional instruments for measuring greenhouse gas concentrations typically employ a singular measurement method, requiring multiple instruments for measurements of different gases, resulting in cumbersome equipment, high power consumption and inconvenient portability. By providing at least two types of laser beams for simultaneous measurement of various gases, it is no longer necessary for numerous devices, further addressing the issue of equipment complexity and rendering the instrument conveniently portable.

In some embodiments, the gas absorption cell 100 is a Herriott cell. And, the gas absorption cell 100 is a multi-pass cell, capable of simultaneously receiving two types of laser beams, and forming at least two separate (or independent) optical paths. For instance, one type of laser beam may be used for carbon dioxide and water vapor concentration measurements, while another type of laser beam may be used for methane, nitrous oxide, and water vapor concentration measurements, thereby accomplishing simultaneous measurements of a variety of gases. A Herriott cell is equipped with two reflective mirrors, and the at least two types of laser beams can be simultaneously reflected between the two reflective mirrors to increase the lengths of the optical paths for interaction between the gas and the laser beam, thereby enhancing the intensity and detection sensitivity of the absorption signal.

In some embodiments, the electronic system unit 200 may include components of the instrument such as the circuit board, the thermoelectric cooler (TEC), and so on. In operation, the electronic system unit 200 will generates a substantial amount of heat, causing an elevation of the internal temperature of the instrument that necessitates additional cooling. The disclosure ingeniously recycles the heat via a heat recovery unit back to the portions of the optical unit that require heat, such as the cell tube 130. This not only diminishes the energy consumption required to cool the instrument, but also reduces the energy consumption required to heat the gas within the cell tube 130, thereby achieving an effect of low power consumption.

The electronic system unit 200 may communicate with various types of external devices through some communication interfaces. The communication interfaces may include an RS232 interface, a USB interface, an SD card slot, a network interface, a Wi-Fi module, a Bluetooth module, and so forth.

In some embodiments, the instrument uses a built-in battery as the energy source. In such situations, since the power consumption is reduced, the battery life and use time can be extended, which is suitable for long-term field use.

The instrument of embodiments of the disclosure ingeniously employs multiple separate optical paths sharing a pair of reflective mirrors (that is, sharing the first reflective mirror 110 and the second reflective mirror 120). The entire device requires only a single gas absorption cell (Herriott Cell) to quickly and accurately measure various different gases simultaneously.

This simplifies the structure of the instrument, eliminates the need for multiple devices for detection, provides a structural groundwork for portability, allowing for the integration of all units into a single carrier 500 for ease carrying.

The instrument of the disclosure incorporates a Herriot Cell into a portable device for greenhouse gas measurement with low power consumption, which exhibits high frequency, remarkable precision, steadfast reliability, and superior portability. It is capable of simultaneous measurement of multiple greenhouse gases, such as carbon dioxide, methane, nitrous oxide, and water vapor. It can accurately measure the concentration of greenhouse gases under various environmental conditions, especially applicable in field settings. Moreover, it facilitates data exchange and control with users via multiple interactive methods.

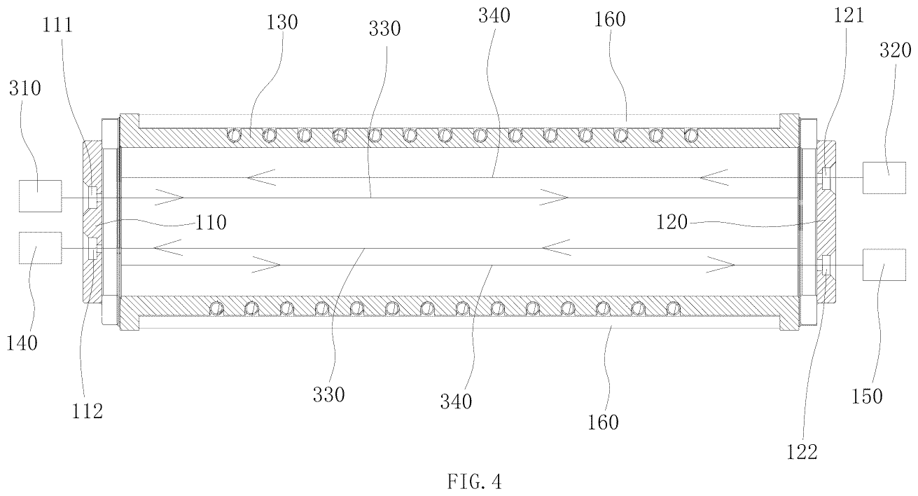

As shown in FIG. 4, the laser unit may include a first laser 310 and a second laser 320. The first laser 310 is positioned adjacent to the first reflective mirror 110 or the second reflective mirror 120 and is capable of emitting a laser beam along one optical path into the cell tube 130. The second laser 320 is positioned adjacent to the first reflective mirror 110 or the second reflective mirror 120 and is capable of emitting a laser beam along another optical path into the cell tube 130.

In the embodiments as shown in the drawings, the first laser 310 is positioned at the rear side of the first reflective mirror 110 (adjacent to the first reflective mirror 110 and outside the cell tube 130), and the second laser 320 is positioned at the rear side of the second reflective mirror 120 (adjacent to the second reflective mirror 120 and outside the cell tube 130).

It should be understood that both the first laser 310 and the second laser 320 can be positioned at the rear side of the same one reflective mirror, either the first reflective mirror 110 or the second reflective mirror 120 (adjacent to the same one reflective mirror and outside the cell tube 130).

In the embodiments shown in the drawings, the laser unit provides two types of laser beams of different wavelengths. However, the mode providing more than two types of laser beams should also be within the protection scope of the disclosure.

For instance, one of the first laser 310 and the second laser 320 may emit a near-infrared laser beam for measuring the concentrations of carbon dioxide and water vapor, while the other one may emit mid-infrared quantum cascade laser beam with a wavelength in the vicinity of 7.4 um for measuring the concentrations of methane, nitrous oxide, and water vapor. Furthermore, the selection of laser sources and wavelengths for the first laser apparatus 310 and the second laser apparatus 320 can be made according to practical needs to measure respective gases.

In some further embodiments, in the internal portion of the gas absorption cell 100, comparisons can be conducted when measuring water vapor, as both the two different type of laser beams are utilized for measuring the concentration of water vapor. The instrument can compare the results of the two-path laser beam for water vapor concentration measurement, thereby verifying the accuracy and consistency of the measurements.

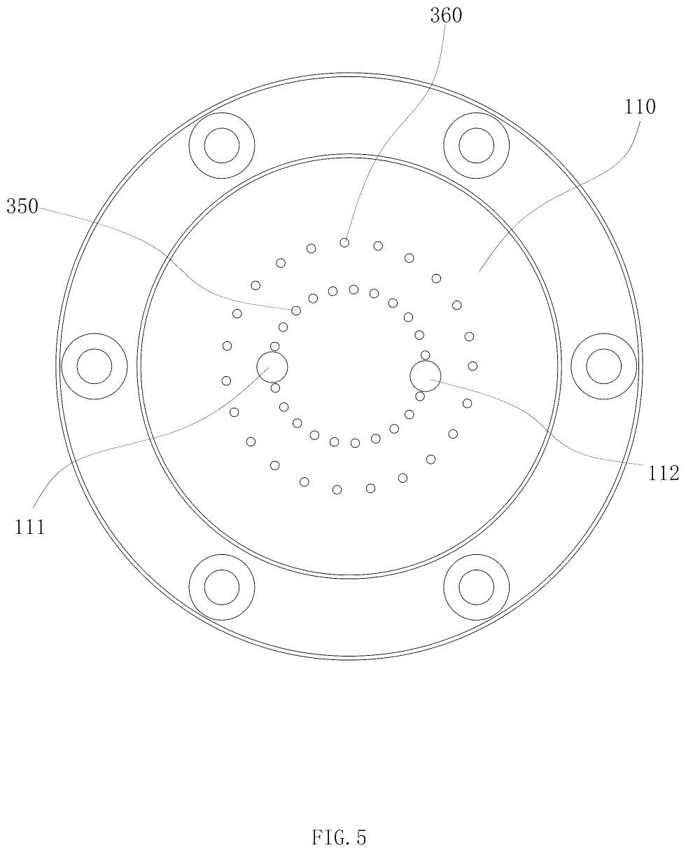

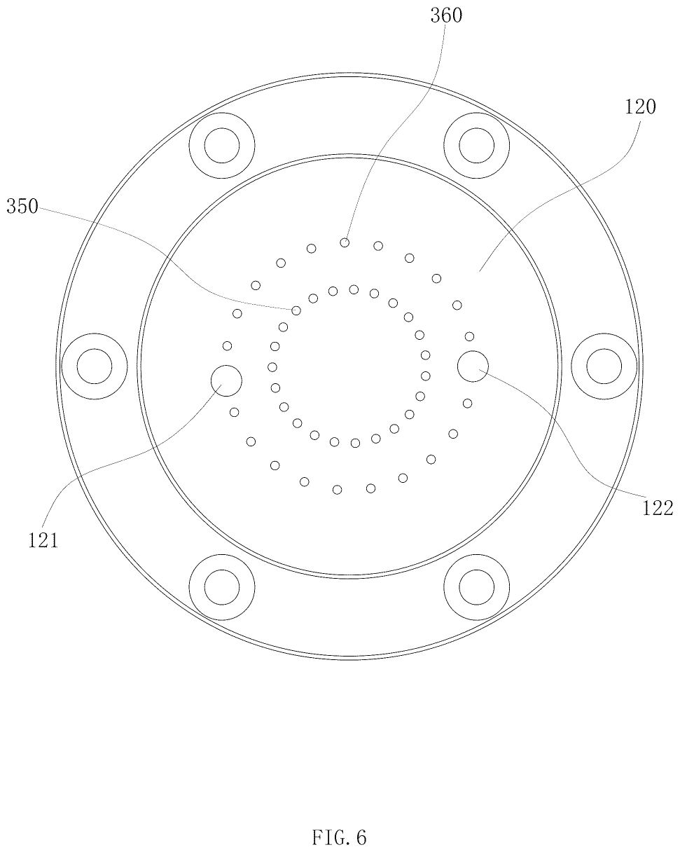

In some further embodiments, the first reflective mirror 110 and the second reflective mirror 120 are both provided with a first reflective portion and a second reflective portion distributed in concentric circles. The first reflective portion of the first reflective mirror and the first reflective portion of the second reflective mirror are composed of a plurality of first reflective points 350 arranged in a ring, and the second reflective portion of the first reflective mirror and the second reflective portion of the second reflective mirror are composed of a plurality of second reflective points 360 arranged in a ring, as shown in FIGS. 5 and 6.

The first reflective portion of the first reflective mirror and the first reflective portion of the second reflective mirror are arranged correspondingly, the laser beam emitted by the first laser 310 is reflected back and forth between the first reflective portion of the first reflective mirror and the first reflective portion of the second reflective mirror to form a first optical path 330, which is a trajectory path formed by the reciprocating reflection of laser beam.

Similarly, the second reflective portion of the first reflective mirror and the second reflective portion of the second reflective mirror are arranged correspondingly, the laser beam emitted by the second laser 320 is reflected back and forth between the second reflective portion of the first reflective mirror and the second reflective portion of the second reflective mirror to form a second optical path 340, which is a trajectory path formed by the reciprocating reflection of laser beam.

The first reflective mirror 110 (as shown in FIG. 5) and the second reflective mirror 120 (as shown in FIG. 6) have similar structures. The mirror surface of each of the first reflective mirror 110 and second reflective mirror 120 respectively includes multiple reflection points (which can be regarded as light spot points) arranged in two concentric circles. Reflection points arranged in one circle forms the first reflective portion, and reflection points arranged in the other circle forms the second reflective portion.

The first reflective portion of the first reflective mirror 110 corresponds to the first reflective portion of the second reflective mirror 120. One laser beam is reflected back and forth between the first reflective portion of the first reflective mirror and the first reflective portion of the second reflective mirror and forms the first light path 330.

Similarly, the second reflective portion of the first reflective mirror 110 corresponds to the second reflective portion of the second reflective mirror 120. Another laser beam is reflected back and forth between the second reflective portion of the first reflective mirror and the second reflective portion of the second reflective mirror and forms the second light path 340.

Through the aforementioned configuration, it is possible to simultaneously form two optical path systems within a single Herriott cell and receive two types of laser beams for concurrent measurement of multiple kinds of gases. This obviates the need for an abundance of equipment, effectively resolving the issue of cumbersome apparatus, thereby rendering this instrument conveniently portable.

As shown in FIGS. 5 and 6, the first reflective mirror 110 is provided with a first light entrance hole 111 and a first light exit hole 112, the second reflective mirror 120 is provided with a second light entrance hole 121 and a second light exit hole 122.

The laser beam emitted by the first laser 310 enters the cell tube 130 from the first light entrance hole 111 to form the first optical path 330 and exits from the first light exit hole 112.

The laser beam emitted by the second laser 320 enters the cell tube 130 from the second light entrance hole 121 to form the second optical path 340 and exits from the second light exit hole 122.

In the embodiment shown in FIGS. 4-6, the first laser 310 and the second laser 320 are positioned at rear sides of different reflective mirrors respectively.

As described above, in some other embodiments, both the first laser 310 and the second laser 320 can be positioned at the rear side of the same one reflective mirror, either the first reflective mirror 110 or the second reflective mirror 120 (adjacent to the same one reflective mirror and outside the cell tube 130).

Accordingly, the first light entrance hole 111 for the first laser 310 can be arranged either on the first reflective mirror 110 or the second reflective mirror 120. Similarly, the second light entrance hole 121 for the second laser 320 can be arranged either on the first reflective mirror 110 or the second reflective mirror 120.

As shown in FIG. 4, the optical unit further includes a first detector 140 and a second detector 150.

The first detector 140 is located on an optical path of the laser beam exiting from the first light exit hole 112. The second detector 150 is located on an optical path of the laser beam exiting from the second light exit hole 122.

In the embodiment shown in FIGS. 4-6, the first detector 140 and the second detector 150 are positioned at rear sides of different reflective mirrors respectively.

As described above, in some other embodiments, both the first detector 140 and the second detector 150 can be positioned at the rear side of the same one reflective mirror, either the first reflective mirror 110 or the second reflective mirror 120 (adjacent to the same one reflective mirror and outside the cell tube 130).

Accordingly, the first light exit hole 112 for the first detector 140 can be arranged either on the first reflective mirror 110 or the second reflective mirror 120. Similarly, the second light exit hole 122 for the second detector 150 can be arranged either on the first reflective mirror 110 or the second reflective mirror 120.

For instance, the laser beam emitted by the first laser 310 is directed towards the first light entrance hole 111 through a reflective mirror 110 or 120. After passing through the first optical path 330, the laser beam exits from the first light exit hole 112. And, when the laser beam exits from the first light exit hole 112, the emitted laser beam is reflected to the first detector 140 through an off-axis parabolic mirror, and the first detector 140 receives the laser beam exiting from the first light exit hole 112.

Similarly, the laser beam emitted by the second laser 320 is directed towards the second light entrance hole 121 through a reflective mirror 120 or 110. After passing through the second optical path 340, the laser beam exits from the second light exit hole 122. And, when the laser beam exits from the second light exit hole 122, the emitted laser beam is reflected to the second detector 150 through an off-axis parabolic mirror, and the second detector 150 receives the laser beam exiting from the second light exit hole 122.

In some embodiments, as shown in FIG. 4, a heating element 160 can be arranged on the cell tube 130. The heating element 160 can be an electrical heating filament, an electrical heating tube, an electrical heating plate, or other kinds of electrical heating components. In some further embodiments, the heating element 160 can be designed to encircle and envelop the cell tube 130, as shown in FIG. 4.

If the heat recycled by the heat recovery unit from the electronic system unit 200 back into the cell tube 130 is insufficient to elevate the temperature of the gas within the cell tube 130 to a desired level, the temperature of the gas within the cell tube 130 can be controlled by performing heating through the heating element 160.

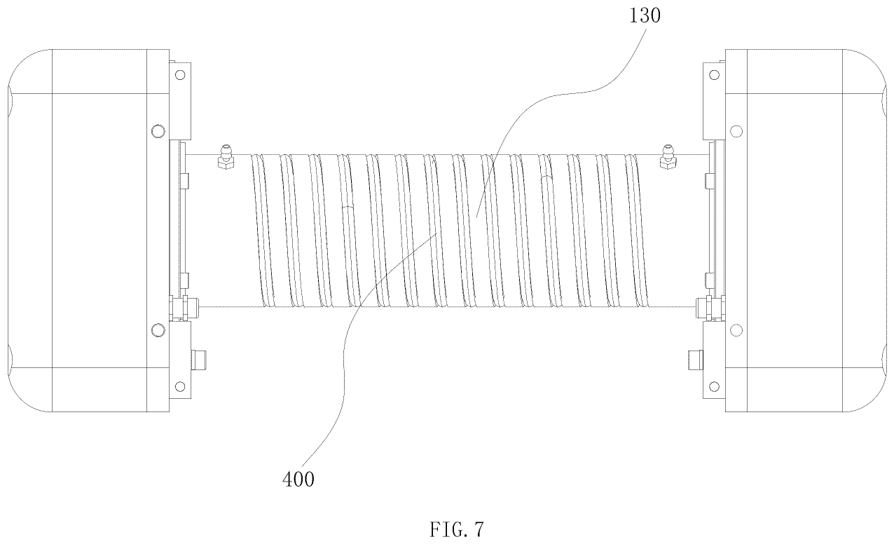

In some embodiments, as shown in FIG. 7, the heat recovery unit may include a circulating water pipe 400. A section of the circulating water pipe 400 is arranged adjacent to or in contact with the electronic system unit 200, while another section of the circulating water pipe 400 is attached to or wound around the cell tube 130. To facilitate the representation of the circulating water pipe 400, the heating element 160 is not shown in FIG. 7.

The heat recovery unit may further include a water pump (not shown). Water resides in the circulating water pipe 400, the water pump drives the water to circulate and flow within the circulating water pipe 400.

When the water passes by the electronic system unit 200, which includes elements such as circuit board and TEC temperature control that generate substantial heat, it can produce the effect of water cooling and heat dissipation. Consequently, the temperature of the water within the circulating water pipe 400 elevates at this time because of heat absorption.

When the water flows to the section of the circulating water pipe 400 that is attached to or wound around the cell tube 130, the gas in the cell tube 130 can be heated by the recovered heat, thereby maintaining the stability of the temperature in the cell tube 130.

This not only solves the problem of heat dissipation and cooling of the electronic system unit 200, but also solves the problem of gas heating in the cell tube 130, which is equivalent to reducing the power consumption of both the two partial systems, and ensures the measurement stability of the instrument at different ambient temperatures, thereby improving the measurement accuracy of the instrument.

As shown in FIG. 1-3, in some further embodiments, the portable instrument may further include a carrier 500 that can be closed. The optical unit, the laser unit, the electronic system unit 200 and the heat recovery unit are all arranged in the carrier 500. When the carrier 500 is closed, the optical unit, the laser unit, the electronic system unit 200 and the heat recovery unit are all in an enclosed space formed by the carrier 500.

In some embodiments, the carrier (500) is a sealable carrier, and the enclosed space is an airtight space.

In some embodiments, the enclosed space is a non-airtight space.

The carrier 500 might be a suitcase or a box or a container. The presence of the carrier 500 not only further enhances the portability of the entire instrument, but also provide an effect of being enclosed for all units, isolating them from the influence of the ambient temperature, dust, etc. Therefore, due to its portability, low power consumption, and being enclosed, the instrument of the disclosure can be used in outdoor or field environments.

For instance, the carrier 500 (suitcase) has the characteristics of water-sealed (watertight), is capable of working in a harsh field environment, adapts to the temperature range from −20 degrees Celsius to 70 degrees Celsius. And in the instrument, the gas sample is drawn into the cell tube 130 of the gas absorption cell 100 through an air pump 610 for measurement.

In some further embodiments, the instrument may be applicable for a variety of interaction manners, including Bluetooth, wireless, wired networking, and USB storage. In these manners, a user can perform operations such as data exchange, control, and data storage with the instrument.

In some further embodiments, the carrier 500 is provided with an air inlet 510 and an air outlet 520. The air inlet 510 or the air outlet 520 is provided with a cooling fan 530. A water cooling radiator 540 is arranged in the carrier 500, and the water cooling radiator 540 is connected with an air inlet 510 and an air outlet 520.

The instrument employs a method of cooling that utilizes water cooling radiator 540 and cooling fan 530 to cool down, so that the temperature inside the carrier 500 will not be too high. Further, the water cooling radiator 540 and the components inside the instrument are also relatively sealed.

In some further embodiments, the portable instrument further includes a calibration unit. The calibration unit includes an air pump 610 and a pressure controller 620.

The air pump 610 is connected to the interior space of the cell tube 130 through a pipeline and can inflate or deflate the cell tube 130. And the pressure controller 620 is arranged on the pipeline and is used for controlling the air pressure inside the cell tube 130.

The calibration unit can calibrate the measurement accuracy of the optical unit. Further, the calibration unit can realize a self-calibration effect of the optical unit by vacuuming periodically or at specific timings. The air pump 610 is used for pumping the gas outside the cell tube 130 into the cell tube 130 or discharging the gas in the cell tube 130. And the pressure controller 620 can control the air pressure inside the cell tube 130.

During normal operation, the air pump 610 pumps the external gas into the cell tube 130 for measurement. And the air pressure inside the cell tube 130 is stably controlled by the pressure controller 620, so as to ensure the stability and accuracy of the measurement.

When calibration is required, the pressure in the cell tube 130 is set to zero through the pressure controller 620, and the air pump 610 vacuums the cell tube 130, so that there is no gas molecule exit inside the cell tube 130 in theory, and the gas concentration is proximate to or equal to zero. At this point, a calibrated zero point can be obtained by measurement taken by the optical unit.

Hereinafter, a method of using the instrument of the disclosure is described by exemplifying the observation of greenhouse gas emission fluxes in the field soil.

Firstly, a gas chamber is installed above the field soil, and closing and opening operations are carried out at different times.

When the gas chamber is closed (or sealed), the gas within the gas chamber is pumped into the cell tube 130 by the air pump 610.

The laser unit emits a near-infrared laser beam for measuring the concentration of carbon dioxide and water vapor, as well as a mid-infrared quantum cascade laser beam with a wavelength in the vicinity of 7.4 um for measuring the concentration of methane, nitrous oxide and water vapor.

The concentration of greenhouse gas can be measured through the first detector 140 and the second detector 150.

And thus, the greenhouse gas emission flux of the soil can be calculated accordingly.

It should be noted that all directional indications in the embodiments of the disclosure (such as up, down, left, right, front, rear, . . . ) is only used to explain the relative position relationship between the parts under a specific attitude (as shown in the attached drawing), the movement, etc., and if the specific attitude changes, the directional indication will change accordingly.

In addition, descriptions of the disclosure involving “first”, “second”, “a”, etc., are for descriptive purposes only and cannot be construed as indicating or implying their relative importance or implying the number of technical features indicated. Thus, features defined by the “first” and “second” may explicitly or implicitly include at least one of those features. In the description of the disclosure, “plurality” means at least two, such as two, three, etc., unless otherwise expressly and specifically qualified.

In the disclosure, unless otherwise expressly specified or qualified, the terms “connected”, “fixed”, etc., shall be construed broadly. For example, “fixed” may be a fixed connection, a detachable connection, or integrated as a whole; the term “connected” may represents mechanically or electrically connected, it can be directly connected or indirectly connected via an intermediate medium, it may be an internal connection between two elements or an interaction between two elements, unless otherwise expressly specified. For one of ordinary skill in the art, the specific meaning of the above terms in the disclosure may be understood according to the specific circumstances.

In addition, the technical solutions of the various embodiments in the disclosure may be combined with each other. The combination shall be capable of being realized by one skilled in the art. When the combination of technical solutions contradicts each other or cannot be realized, it shall be considered that such a combination of technical solutions does not exist, and is not within the scope of protection claimed by the disclosure.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A portable instrument for measuring gas concentration comprising:
a laser unit configured to provide a laser beam required for laser spectral absorption;
an optical unit comprising a gas absorption cell, the gas absorption cell comprises a first reflective mirror, a second reflective mirror and a cell tube, the first reflective mirror and the second reflective mirror are respectively connected to opposite ends of the cell tube, when the laser unit emits a laser beam, an optical path is formed between the first reflective mirror and the second reflective mirror within the cell tube;
an electronic system unit;
a heat recovery unit configured to recycle heat generated by the electronic system unit to the optical unit.

2. The portable instrument for measuring gas concentration according to claim 1, wherein the heat recovery unit comprises a circulating water pipe, a section of the circulating water pipe is arranged adjacent to or in contact with the electronic system unit, and another section of the circulating water pipe is attached to or wound around the cell tube.

3. The portable instrument for measuring gas concentration according to claim 1 further comprising a closable carrier, wherein the optical unit, the laser unit, the electronic system unit and the heat recovery unit are all arranged in the closable carrier,
when the closable carrier is closed, the optical unit, the laser unit, the electronic system unit and the heat recovery unit are all in an enclosed space.

4. The portable instrument for measuring gas concentration according to claim 3, wherein the closable carrier is a sealable carrier, and the enclosed space is an airtight space.

5. The portable instrument for measuring gas concentration according to claim 3, wherein the enclosed space is a non-airtight space.

6. The portable instrument for measuring gas concentration according to claim 3, wherein the closable carrier is provided with an air inlet and an air outlet, the air inlet or the air outlet is provided with a cooling fan, the closable carrier has a water cooling radiator arranged internally, the water cooling radiator is connected with the air inlet and the air outlet.

7. The portable instrument for measuring gas concentration according to claim 1, wherein the cell tube is provided with a heating element.

8. The portable instrument for measuring gas concentration according to claim 1, wherein
the laser unit is configured to provide at least two laser beams of different wavelengths;
when the laser unit emits at least two laser beams, at least two separate optical paths are formed between the first reflective mirror and the second reflective mirror within the cell tube.

9. The portable instrument for measuring gas concentration according to claim 8, wherein
the laser unit includes a first laser and a second laser,
the first laser is positioned adjacent to the first reflective mirror or the second reflective mirror and is capable of emitting a laser beam along one optical path into the cell tube,
the second laser is positioned adjacent to the first reflective mirror or the second reflective mirror and is capable of emitting a laser beam along another optical path into the cell tube.

10. The portable instrument for measuring gas concentration according to claim 9, wherein
the first reflective mirror and the second reflective mirror are both provided with a first reflective portion and a second reflective portion distributed in concentric circles, the first reflective portion of the first reflective mirror and the first reflective portion of the second reflective mirror are composed of a plurality of first reflective points arranged in a ring, the second reflective portion of the first reflective mirror and the second reflective portion of the second reflective mirror are composed of a plurality of second reflective points arranged in a ring;
the first reflective portion of the first reflective mirror and the first reflective portion of the second reflective mirror are arranged correspondingly, the laser beam emitted by the first laser is reflected back and forth between the first reflective portion of the first reflective mirror and the first reflective portion of the second reflective mirror to form a first optical path;
the second reflective portion of the first reflective mirror and the second reflective portion of the second reflective mirror are arranged correspondingly, the laser beam emitted by the second laser is reflected back and forth between the second reflective portion of the first reflective mirror and the second reflective portion of the second reflective mirror to form a second optical path.

11. The portable instrument for measuring gas concentration according to claim 10, wherein
the first reflective mirror or the second reflective mirror is provided with a first light entrance hole, and the first reflective mirror or the second reflective mirror is provided with a first light exit hole,
the first reflective mirror or the second reflective mirror is provided with a second light entrance hole, and the first reflective mirror or the second reflective mirror is provided with a second light exit hole,
the laser beam emitted by the first laser enters the cell tube from the first light entrance hole to form the first optical path and exits from the first light exit hole,
the laser beam emitted by the second laser enters the cell tube from the second light entrance hole to form the second optical path and exits from the second light exit hole.

12. The portable instrument for measuring gas concentration according to claim 11, wherein the optical unit further comprising:
a first detector located on an optical path of the laser beam exiting from the first light exit hole; and
a second detector located on an optical path of the laser beam exiting from the second light exit hole.

13. The portable instrument for measuring gas concentration according to claim 1, further comprising a calibration unit, the calibration unit comprises an air pump and a pressure controller,
the air pump is connected to an interior space of the cell tube through a pipeline and can inflate or deflate the cell tube,
the pressure controller is arranged on the pipeline and configured to control an air pressure inside the cell tube.
